(19) United States
(12) Patent Application Publication
(10) Pub. No.: US 2024/0307298 A1
(43) Pub. Date: Sep. 19, 2024

(54) PROCESS FOR MANUFACTURING AN OIL-AN-WATER EMULSION WITH A LOW PFAT5 VALUE IN ADMIXTURES FOR PARENTERAL NUTRITION

(71) Applicant: Fresenius Kabi Deutschland GmbH, Bad Homburg (DE)

(72) Inventors: Edmundo BRITO DE LA FUENTE, Friedrichsdorf (DE); Crispulo GALLEGOS-MONTES, Bad Homburg (DE); Lida A. Quinchia-Bustamante, Bad Homburg (DE); Getachew WELDEABEZGI, Oberursel (DE); Mihaela TURCANU, Bad Homburg (DE); Nadja Siegert, Bad Homburg (DE)

(21) Appl. No.: 18/571,198

(22) PCT Filed: Jun. 14, 2022
(86) PCT No.: PCT/EP2022/066086

§ 371 Date: Dec. 15, 2023

(30) Foreign Application Priority Data
Jun. 16, 2021 (EP) 21179768.3

(51) Int. Cl. A61K 9/00; A61K 9/107; A61K 31/7004; A61K 47/10; A61K 47/12; A61K 47/18; A61K 47/22; A61K 47/24; A61K 47/44

(52) U.S. Cl. CPC A61K 9/0029; A61K 9/107; A61K 31/7004; A61K 47/10; A61K 47/12; A61K 47/183; A61K 47/22; A61K 47/24; A61K 47/44

(57) ABSTRACT

The invention relates to a process for manufacturing oil-in-water emulsions with a PFATs value that remains below 0.05% for at least 24 hours, preferably for at least 48 hours after admixing the emulsions with an amino acid and a glucose solution suitable for parenteral administration.

### FIELD OF THE INVENTION

The invention relates to a process for manufacturing oil-in-water emulsions for parenteral administration.

### BACKGROUND OF THE INVENTION

Lipid emulsions have long been used for providing parenteral nutrition, and a number of high-quality oil-in-water emulsions are commercially available for this purpose.

However, these emulsions are often administered in admixture with an amino acid solution and/or a glucose solution. The stability of these admixtures, i.e. the stability of oil-in-water emulsions after their dilution with an amino acid solution and/or a glucose solution has remained a challenge.

Compendial requirements include the determination of several parameters in order to warrant requisite stability.

One of these parameters is the PFAT₅ value (percentage of fat residing in oil droplets larger than 5 μm in diameter; USP <729>) of the oil-in-water emulsion.

Upon admixture with an amino acid solution and/or a glucose solution the PFAT₅ value should remain below 0.05% for at least 24 hours, preferably for at least 48 hours.

### DESCRIPTION OF THE INVENTION

The present invention relates to a process for manufacturing an oil-in-water emulsion comprising a water phase and 5 to 25 wt. % of an oil phase based on the total weight of the emulsion, the process comprising the following steps:
(a) providing an oil phase comprising one or more oils selected from the group consisting of animal derived oils, plant-derived oils, fungal oils, synthetic or semi-synthetic fatty acid triglycerides, microbial oils and algae oils,
(b) providing an aqueous phase 1 comprising water,
(c) obtaining a pre-emulsion by mixing the oil phase provided in step a) with the aqueous phase 1 provided in step b),
(d) obtaining a first emulsion by homogenizing the pre-emulsion obtained in step c) by means of at least one counter-jet disperser, wherein the homogenization is performed in 2-6 cycles, at a pressure of 600-1800 bar and at a temperature of 40-80° C.,
(e) providing an aqueous phase 2 comprising water,
(f) obtaining the oil-in-water emulsion by mixing the first emulsion obtained in step d) with the aqueous phase 2 provided in step e) and
(g) sterilizing the oil-in-water emulsion obtained in step f) and filling it into a suitable container either before or after sterilization,
   wherein in step a) or b) a pharmaceutically acceptable emulsifier, characterized in that it comprises at least 70 wt. % phosphatidyl choline based on the total weight of the emulsifier, is added,
   wherein in the pre-emulsion obtained in step c) and in the first emulsion obtained in step d) the concentration of the oil phase is 130 to 350% of the concentration of the oil phase in the emulsion obtained in step f),
   and wherein for 24 hours, preferably 48 hours, after mixing the oil-in-water emulsion with an amino acid solution suitable for parenteral administration and/or a glucose solution suitable for parenteral administration the PFAT₅ value of the resulting mixture does not exceed 0.05%.

### The Oil Phase

The oil-in-water emulsions manufactured according to the process of the present invention comprise 5 to 25 wt. %, e.g. 10 wt. % or 20 wt. %, of an oil phase based on the total weight of the emulsion.

The oil phase comprises one or more oils selected from the group consisting of animal-derived oils, plant-derived oils, fungal oils, synthetic or semi-synthetic fatty acid triglycerides, microbial oils and algae oils.

Preferably, the oil phase comprises one or more oils selected from the group consisting of soybean oil, olive oil, fish oil, medium chain triglycerides and structured lipids.

The term “fish oil” refers to “purified fish oil” and to “purified fish oil rich in omega 3 fatty acids”, the latter according to the European Pharmacopoeia 6.0 comprising at least 9% (w/w) of the omega-3-fatty acid docosahexaenoic acid (DHA) and at least 13% (w/w) of the omega-3 fatty acid eicosapentaenoic acid (EPA) expressed as triglycerides. Fish oils are commercially available.

In the context of the present disclosure the term “fish oil” also refers to fish oil extracts that may be further enriched or downgraded respectively in certain fatty acids. Such fish oil extracts are commercially available, e.g. from Solutex S.L.

The term “medium chain triglycerides” (MCT) refers to triglycerides of fatty acids having 6 to 12 carbon atoms in length, including caproic acid, caprylic acid, capric acid and lauric acid. MCT are commercially available.

The term “structured lipids” refers to purified structured triglycerides that can be defined as an inter-esterified mixture of equimolar amounts of long chain (fatty acid) triglycerides and medium chain triglycerides (MCT), corresponding to 60 to 68 wt. %, preferably 64 wt. % and 32 to 40 wt. %, preferably 36 wt. %, respectively. The fatty acids are randomly distributed within the inter-esterified triglyceride molecule. Purified structured triglycerides mainly consist of mixed chain triglycerides. Structured lipids are commercially available.

In a preferred embodiment the oil phase comprises soybean oil.

In another preferred embodiment the oil phase comprises structured lipids.

In another preferred embodiment the oil phase comprises soybean oil, MCT, olive oil and fish oil.

In further preferred embodiments, the oil phase comprises olive oil and fish oil, olive oil and soybean oil, soybean oil and fish oil, olive oil and MCT or soybean oil and MCT.

Where the oil phase comprises soybean oil, it is preferably present in amounts of 25 to 100 wt. % based on the total weight of the oil phase. It may for example be comprised in amounts of 25 to 35 wt. %, 45 to 55 wt. % or 90 to 100 wt. % based on the total weight of the oil phase.

Where the oil phase comprises olive oil, it is preferably present in amounts of 10 to 60 wt. %, preferably 20 to 50 wt. % based on the total weight of the oil phase. It may for example be present in amounts of 20 to 40 wt. % based on the total weight of the oil phase.

Where the oil phase comprises MCT, it is preferably present in amounts of 25 to 60 wt. %, preferably 30 to 50 wt. % based on the total weight of the oil phase. Where the oil phase comprises fish oil, it is preferably present in amounts of 10 to 50 wt. %, preferably 10 to 30 wt. % based on the total weight of the oil phase. In a particularly preferred embodiment the oil phase comprises 25 to 35 wt. %, preferably 30 wt. %, soybean oil, 25 to 35 wt. %, preferably 30 wt. %, MCT, 20 to 30 wt. %, preferably 25 wt. %, olive oil and 10 to 20 wt. %, preferably 15 wt. %, fish oil based on the total weight of the oil phase.

### The Droplet Size

As the emulsions manufactured according to the process of the present invention are oil-in-water emulsions, the continuous phase is aqueous and comprises oil droplets. These oil droplets are stabilized within the aqueous phase by at least one emulsifier and optionally further additives. The size of the oil droplets depends on the qualitative and quantitative composition of the emulsion and its preparation.

The oil droplets of the emulsions manufactured according to the process of the present invention preferably have a mean diameter (volume based) of 130 to 450 nm, preferably 150 to 400 nm, more preferably 180 to 350 nm, when measured directly upon sterilization using a Mastersizer 3000 (Malvern) according to USP <729>.

### The PFAT₅ Value

According to the USP in an oil-in-water emulsion for parenteral administration the percentage of fat residing in oil droplets larger than 5 μm in diameter (PFAT₅ value) must not exceed 0.05%.

Where an emulsion for parenteral administration is mixed with an amino acid solution and/or a glucose solution before administration, the PFAT₅ value should remain below 0.05% for at least 24 hours, preferably for at least 48 hours after the emulsion has been mixed with the amino acid solution and/or the glucose solution.

The PFAT₅ value is measured according to one of the methods according to USP <729>.

The emulsions manufactured according to the process of the present invention have a PFAT₅ value below 0.05%, preferably below 0.04%, more preferably below 0.3%. The PFAT₅ value remains below 0.05%, preferably below 0.04%, more preferably below 0.03%, during the shelf life of the emulsions. The shelf life of the emulsions is preferably at least 1 year, more preferably at least 1.5 years, more preferably at least 2 years, when stored at 5° C. to 25° C. at a relative humidity of 40 to 60%.

The PFAT₅ value of the emulsions manufactured according to the process of the present invention remains below 0.05% for at least 24 hours, preferably for at least 48 hours, after they have been mixed with an amino acid solution suitable for parenteral administration and/or a glucose solution suitable for parenteral administration, preferably with an amino acid solution suitable for parenteral administration and a glucose solution suitable for parenteral administration.

The PFAT₅ value of the emulsions manufactured according to the process of the present invention remains below 0.05% for at least 24 hours, preferably for at least 48 hours, after they have been mixed with an amino acid solution suitable for parenteral administration, preferably comprising 5 to 15% (w/v) amino acids based on the total volume of the amino acid solution, preferably at a volume ratio of emulsion to amino acid solution of 1 to 1.0-6.1, more preferably 1 to 1.1-4.6, and/or a glucose solution suitable for parenteral administration, preferably comprising 5 to 45% (w/v) glucose based on the total volume of the glucose solution, preferably at a volume ratio of 1 to 1.3-7.0, more preferably 1 to 1.4-4.0, wherein the pH of the amino acid solution is preferably between 5.0 and 6.5 and the osmolarity of the amino acid solution is preferably between 500 mOsmol/L and 1200 mOsmol/L, and the pH of the glucose solution is preferably between and 3.5 to 6.5 and the osmolarity of the glucose solution is preferably between 500 and 2000 mOsmol/L.

The PFAT₅ value of the oil-in-water emulsions manufactured according to the process of the present invention remains below 0.05% for at least 24 hours, preferably for at least 48 hours, after they have been mixed with an amino acid solution suitable for parenteral administration, preferably comprising 5 to 15% (w/v) amino acids based on the total volume of the amino acid solution, and a glucose solution suitable for parenteral administration, preferably comprising 5 to 45% (w/v) glucose based on the total volume of the glucose solution, preferably at a weight ratio of emulsion to glucose solution to amino acid solution of 1 to 1.3-7.0 to 1.0-6.1, more preferably 1 to 1.1-4.6 to 1.4-4.0, wherein preferably the pH of the mixture is between 4.5 and 6.5, more preferably between 5.0 and 6.0, and wherein the osmolarity of the mixture is preferably between 700 and 1550 mOsmol/L.

In one embodiment the PFAT₅ value of the oil-in-water emulsion manufactured according to the process of the present invention remains below 0.05% for at least 24 hours, preferably at least 48 hours, after it has been mixed with a glucose solution suitable for parenteral administration comprising 11 to 13% (w/v) glucose based on the total volume of the glucose solution and an amino acid solution suitable for parenteral administration comprising 10 to 12% (w/v) amino acids based on the total volume of the amino acid solution at a volume ratio of emulsion to glucose solution to amino acid solution of 1 to 3.2-3.9 to 1.1-2.3, wherein the pH of the mixture is between 5.0 and 6.0, and wherein the osmolarity of the mixture is between and 700 and 1000 mOsmol/L.

In another embodiment the PFAT₅ value of the oil-in-water emulsion manufactured according to the process of the present invention remains below 0.05% for at least 24 hours, preferably for at least 48 hours, after it has been mixed with a glucose solution suitable for parenteral administration comprising 18 to 22% (w/v) glucose based on the total volume of the glucose solution and an amino acid solution suitable for parenteral administration comprising 6 to 12% (w/v) amino acids based on the total volume of the amino acid solution at a volume ratio of emulsion to glucose solution to amino acid solution of 1 to 2.6-7.0 to 1.5-6.1, wherein the pH of the mixture is between 5.0 and 6.0, and wherein the osmolarity of the mixture is between 800 and 1100 mOsmol/L.

In another embodiment the PFAT₅ value of the oil-in-water emulsion manufactured according to the process of the present invention remains below 0.05% for at least 24 hours, preferably for at least 48 hours after it has been mixed with a glucose solution suitable for parenteral administration comprising 42% (w/v) glucose based on the total volume of the glucose solution and an amino acid solution suitable for parenteral administration comprising 10% (w/v) amino acids based on the total volume of the amino acid solution at a volume ratio of emulsion to glucose solution to amino acid solution of 1 to 1.3-2.8 to 1.5-4.6, wherein the pH of the mixture is between 5.0 and 6.0, and wherein the osmolarity of the mixture is between and 1300 and 1600 mOsmol/L.

### The Emulsifier

The oil-in-water emulsions manufactured according to the process of the present invention comprise at least one pharmaceutically acceptable emulsifier comprising at least 70 wt. % phosphatidyl choline based on the total weight of the emulsifier. The term “emulsifier” refers to compounds which stabilize the composition by reducing the interfacial tension between the oil phase and the water phase and which typically comprise at least one hydrophobic group and at least one hydrophilic group. These emulsifiers (which may also be referred to as surfactants) are preferably used in amounts effective to provide, optionally together with further surfactants present, a stable and even distribution of the oil phase within the aqueous phase.

The at least one emulsifier comprises at least one phospholipid. Within the meaning of the present disclosure the term “phospholipid” refers to naturally occurring or synthetic phospholipids that may be suitably refined. Suitable phospholipids include, but are not limited to, phospholipids derived from corn, soybean, egg or other animal origin, or mixtures thereof. Phospholipids typically comprise mixtures of diglycerides of fatty acids linked to the choline ester of phosphoric acid and can contain differing amounts of other compounds depending on the method of isolation. Typically, commercial phospholipids are a mixture of acetone-insoluble phosphatides. Preferably, the phospholipids are obtained from egg or other animal origin, or from seeds including soybean and corn, using methods well known in the art. Phospholipids obtained from soybean are referred to herein as soy phospholipids. Phospholipids obtained from egg are referred to herein as egg phospholipids.

The emulsions manufactured according to the process of the present invention comprise phospholipids as emulsifier, more preferably the phospholipids are selected from the group consisting of egg phospholipids, soy phospholipids, and mixtures thereof.

The emulsifier used in the process according to the present invention comprises at least 70 wt. %, e.g. at least 72 wt. %, preferably at least 74 wt. %, more preferably at least 76%, most preferably at least 78 wt. %, phosphatidyl choline based on the total weight of the emulsifier. Such emulsifiers are commercially available.

Preferably, the emulsifier is used in an amount of 0.5 to 5% (w/v), more preferably 0.5 to 3% (w/v), most preferably 1.0 to 2.0% (w/v) based on the total volume of the emulsion.

### The Co-Surfactant

The oil-in-water emulsions manufactured according to the process of the present invention may further comprise a pharmaceutically acceptable co-surfactant.

A co-surfactant is an amphiphilic molecule, i.e. a molecule that contains both hydrophilic and lipophilic groups. Usually, a co-surfactant substantially accumulates with the emulsifier at the interfacial layer. The hydrophile-lipophile balance (HLB) number is used as a measure of the ratio of hydrophilic and lipophilic groups present in a surfactant or co-surfactant, respectively.

Preferably, a co-surfactant with a very low HLB value (thus with a relatively high affinity to oil) is used together with an emulsifier with a high HLB to modify the overall HLB of the system. Unlike the emulsifier, the co-surfactant may not be capable of forming self-associated structures, like micelles, on its own. Several kinds of molecules including nonionic emulsifiers, alcohols, amines and acids, can function as co-surfactants in a given system. The co-surfactant is usually used in a lower amount than that of the emulsifier. Apart from modifying the overall HLB value of the system, the co-surfactant has the effect of further reducing the interfacial tension and increasing the fluidity of the interface. Co-surfactants may also adjust the curvature of the interfacial film by partitioning between the tails of the emulsifier chains, allowing greater penetration of the oil between the emulsifier tails.

Preferably, the co-surfactant is a free long chain fatty acid or a salt thereof, preferably a free unsaturated fatty acid or a salt thereof, preferably an omega-9 fatty acid or a salt thereof, more preferably a monounsaturated omega-9 fatty acid or a salt thereof, more preferably oleic acid or sodium oleate.

The total amount of the co-surfactant is preferably in the range of from 0.01% to 1%, more preferably in the range of from 0.02% to 0.5%, more preferably in the range of from 0.02% to 0.2% based on the total volume of the emulsion (w/v).

### The Tonicity Agent

The oil-in-water emulsions manufactured according to the process of the present invention may comprise at least one pharmaceutically acceptable tonicity agent. Tonicity agents are used to confer tonicity. Suitable tonicity agents may be selected from the group consisting of sodium chloride, mannitol, lactose, dextrose, sorbitol, glycerol and mixtures thereof. Preferably, the tonicity agent is glycerol.

Preferably, the total amount of tonicity agents is in the range of 0.1 to 10%, more preferably from 1% to 5%, more preferably from 1% to 4%, more preferably 1% to 3%, more preferably from 1.5% to 2.8%, and even more preferably from 2.0% to 2.5% based on the total volume of the emulsion (w/v).

In case the tonicity agent is glycerol the preferred amount is 2.0% to 2.8%, the most preferred amount is 2.1% to 2.6% based on the total volume of the emulsion (w/v).

Preferably, the oil-in-water emulsion has an osmolality in the range of 300 to 400 mOsmol/L.

### The Antioxidant

The oil-in-water emulsion manufactured according to the process of the present invention may comprise at least one pharmaceutically acceptable antioxidant. An antioxidant may be any pharmaceutically acceptable compound having antioxidant activity, for example, the antioxidant may be selected from the group consisting of sodium metasulfite, sodium bisulfite, sodium sulfite, sodium thiosulfate, thioglycerol, thiosorbitol, thioglycolic acid, cysteine hydrochloride, n-acetyl-cysteine, citric acid, alpha-tocopherol, beta-tocopherol, gamma-tocopherol, delta-tocopherol, tocotrienols, soluble forms of vitamin E, butylated hydroxyanisole (BHA), butylated hydroxytoluene (BHT), t-butylhydroquinone (TBHQ), monothioglycerol, propyl gallate, histidine, enzymes such as superoxide dismutase, catalase, selenium glutathione peroxidase, phospholipid hydroperoxide and glutathione peroxidase, Coenzyme Q10, carotenoids, quinones, bioflavonoids, polyphenols, bilirubin, ascorbic acid, isoascorbic acid, uric acid, metal-binding proteins, ascorbic acid palmitate, and mixtures thereof. The at least one antioxidant is particularly selected from the group consisting of alpha tocopherol, beta tocopherol, gamma tocopherol, delta tocopherol, tocotrienols, ascorbic acid, and mixtures of two or more thereof. Preferably, the antioxidant is alpha tocopherol or mixture of alpha-, beta- and gamma-tocopherol.

If present, the total amount of agents with antioxidant activity is preferably in the range of from 0.01% to 0.05%, more preferably from 0.01% to 0.04%, more preferably from 0.01% to 0.03%, and even more preferably from 0.015% to 0.025% based on the total volume of the emulsion (w/v).

### The pH Adjusting Agent

The pH of the oil-in-water emulsions manufactured according to the process of the present invention may be adjusted by adding solutions of conventionally known acids or bases such as HCl and NaOH or by using buffers, such as phosphate buffers.

The final pH of the emulsion is preferably in the range of from 7.0 to 10.0, more preferably between 7.5 and 9.5, most preferably between 7.5 and 8.5.

Preferably, the pH of the oil-in-water emulsions manufactured according to the process of the present invention is adjusted using a solution of NaOH.

### The Preservative

The oil-in-water emulsions manufactured according to the process of the present invention may further comprise a pharmaceutically acceptable preservative.

Suitable preservatives are 4-hydroxybenzoic acid as well as salts and esters thereof, sorbic acid as well as salts and derivatives thereof, thiomersal, chlorbutanol, chlorhexidine and salts thereof, phenylmercury salts, p-chlorocresol, ethylenediamine-tetraacetic acid and salts thereof, phenoxyethanol or mixtures thereof.

Typically, the preservative is used in concentrations between 0.001 and 2.0 wt. % based on the total weight of the emulsion.

Preferably, the preservative is ethylenediaminetetraacetic acid or a pharmaceutically acceptable salt thereof.

Where the preservative is ethylenediaminetetraacetic acid or a pharmaceutically acceptable salt thereof, it is preferably used in a concentration of 0.05 to 0.8 wt. %, preferably 0.1 to 0.7 wt. %, based on the total weight of the emulsion.

### Route of Administration

The oil-in-water emulsions manufactured according to the process of the present invention are adapted for parenteral administration. Preferably, the compositions according to the present disclosure are administered intravenously, either into a peripheral or a central vein.

Oil-in-water emulsions for parenteral administration must be sterile, pyrogen-free, well tolerated, free of particulate impurities and storage stable. Their pH should be as close as possible to the pH of the blood.

Step a-Providing the Oil Phase

Step a) is preferably carried out by mixing the oil or oils and optionally a pharmaceutically acceptable antioxidant and/or a pharmaceutically acceptable co-surfactant. This step is preferably carried out by mixing, e.g. by means of an Ultra-Turrax, e.g. at 5000 rpm, e.g. for 5 minutes, at a temperature of 55 to 85° C., e.g. at 60 to 70° C. or to 75 to 85° C., until a homogeneous and clear phase is obtained.

In particular, it is to be understood that the at least one pharmaceutically acceptable emulsifier may be added either in step a) or in step b).

Preferably, where the emulsifier is added in step a) the emulsifier is added after the oil phase has been heated to 55 to 85° C.

Step b—Providing the Aqueous Phase 1

Step b) is preferably carried out by providing water for injection and optionally adding a pharmaceutically acceptable tonicity agent and/or a pharmaceutically acceptable co-surfactant and/or a pharmaceutically acceptable preservative.

Optionally, the pH of the aqueous phase 1 is adjusted to 8.5-10.0, preferably to 9.0 to 10.0.

The aqueous phase is then heated to a temperature of 55 to 85° C., e.g. to 60 to 70° C. or to 75 to 85° C.

In particular, it is to be understood that the at least one pharmaceutically acceptable emulsifier may be added either in step a) or in step b).

Preferably, where the emulsifier is added in step b) the emulsifier is added after the aqueous phase has been heated to 55 to 85° C.

Step c—Obtaining the Pre-Emulsion

The method according to the present invention comprises mixing the oil phase provided in step a) with the aqueous phase 1 provided in step b) thereby forming a pre-emulsion. The mixing may be carried out by any method known to those skilled in the art, e.g. by means of an Ultra-Turrax, e.g. for 5 to 15 minutes, e.g. for 10 to 12 minutes at e.g. 5000 to 15000 rpm, e.g. at 10000 rpm.

Preferably, the oil phase is added to the aqueous phase or vice-versa at a temperature in the range of from 55 to 85° C., e.g. at a temperature between 60 and 70° C. or between 75 and 85° C.

Optionally, the pH of the pre-emulsion may be adjusted to a pH in the range of from 8.5 to 10.0, preferably to pH from 9.0 to 10.0.

Optionally, water for injection is added to compensate for the potential loss of water during processing the pre-emulsion.

In the process according to the present invention the concentration of the oil phase in the pre-emulsion obtained in step c) and in the first emulsion obtained n step d) is higher than the concentration of the oil phase in the emulsion obtained in step f). This is because in step f) the first emulsion obtained in step d) is diluted with the aqueous phase 2 provided in step e).

The concentration of the oil phase in steps c) and d) is at least 130% of the concentration of the oil phase in the emulsion obtained in step f), e.g. 130% to 350%.

Preferably, the concentration of the oil phase in steps c) and d) is at least 150% of the concentration of the oil phase in the emulsion obtained in step f), e.g. 150% to 350%.

More preferably, the concentration of the oil phase in steps c) and d) is at least 180% of the concentration of the oil phase in the emulsion obtained in step f), e.g. 180% to 350%, preferably 180% to 330%.

In a preferred embodiment, the concentration of the oil phase in steps c) and d) is 180% to 330% of the concentration of the oil phase in the emulsion obtained in step f).

In a particularly preferred embodiment, the concentration of the oil phase in steps c) and d) is 180% to 300% of the concentration of the oil phase in the emulsion obtained in step f).

In an even more preferred embodiment, the concentration of the oil phase in steps c) and d) is 200% to 300% of the concentration of the oil phase in the emulsion obtained in step f).

In a particularly preferred embodiment, the concentration of the oil phase in steps c) and d) is 200% of the concentration of the oil phase in the emulsion obtained in step f), i.e. for example if the concentration of the oil phase in the emulsion obtained in step e) is 20 wt. % based on the total weight of the emulsion, the concentration of the oil phase in step c) is 40 wt. % based on the total weight of the pre-emulsion obtained in step c) and of the first emulsion obtained in step d).

In another particularly preferred embodiment, the concentration of the oil phase in steps c) and d) is 250% of the concentration of the oil phase in the emulsion obtained in step e), i.e. for example if the concentration of the oil phase in the emulsion obtained in step f) is 20 wt. % based on the total weight of the emulsion, the concentration of the oil phase in step c) is 50 wt. % based on the total weight of the pre-emulsion obtained in step c) and of the first emulsion obtained in step d).

Step d—Obtaining the First Emulsion

In step d) of the process according to the present invention the pre-emulsion obtained in step c) is homogenized by means of at least one counter jet disperser, preferably for 2 to 6 cycles at a pressure of 600 to 1800 bar, more preferably at a pressure of 800 to 1400 bar, and preferably at a temperature of 40 to 80° C., more preferably at a temperature of 45 to 65° C., most preferably at a temperature of 50 to 60° C.

Where the oil phase comprises soybean oil, in step d) the pre-emulsion obtained in step c) is homogenized by means of at least one counter jet disperser preferably in 3 to 4 cycles at a pressure of 1200 to 1400 bar and at a temperature of 45 to 65° C., preferably 50 to 60° C.

Where the oil phase comprises soybean oil, MCT, olive oil and fish oil, in step d) the pre-emulsion obtained in step c) is homogenized by means of at least one counter jet disperser preferably in 3 to 4 cycles at a pressure of 800 to 1000 bar and at a temperature of 45 to 65° C., preferably 50 to 60° C.

Optionally, in step d) the pH is adjusted to values between 8.5 and 10.0, preferably to values between 9.0 and 10.0.

Step e—Providing the Aqueous Phase 2

Step e) is preferably carried out by providing water for injection and optionally adding a pharmaceutically acceptable tonicity agent and/or a pharmaceutically acceptable co-surfactant and/or a pharmaceutically acceptable preservative.

Optionally, the pH of the aqueous phase 2 is adjusted to 8.5 to 10.0, preferably to 9.0 to 10.0.

Step f—Obtaining the Emulsion

The method according to the present invention comprises mixing the first emulsion obtained in step d) with the appropriate amount of aqueous phase 2 provided in step e) to obtain the oil-in-water emulsion with desired concentration of oil phase being 5 to 25 wt. % based on the total weight of the emulsion.

Preferably, the first emulsion obtained in step d) is cooled to 20 to 40° C. before it is mixed with the water phase 2.

Optionally, the pH of the emulsion is adjusted to 8.5 to 10.0, preferably to 9.0 to 10.0.

Step g—Sterilizing the Emulsion

The method further comprises the sterilization of the oil-in-water emulsion obtained in step f) to ensure its suitability for parenteral administration.

The sterilization may be carried out by any suitable method known to those skilled in the art.

Preferably, the sterilization is carried out by autoclaving, preferably at a temperature in the range of from 119 to 122° C., more preferably at a temperature around 121° C., preferably for 1 minute to 30 minutes, preferably for 10 to 15 minutes.

### EXAMPLES

### Example 1

Different emulsions were prepared from the ingredients listed in table 1. Their composition only differed in the choice of the emulsifier (egg yolk lecithin) which was either PL1 (not according to the invention) or PL2 (table 2).

TABLE 1

| ingredient | amount (g) |
|---|---|
| glycerol | 44.00 |
| emulsifier (either PL1 or PL2) | 24.00 |
| NaOH 1M | q.s. |
| soybean oil | 400.00 |
| WFI | ad 2000 |
| nitrogen | q.s. |

TABLE 2

Composition (% w/w)

| Component | PL1 (comparative) | PL2 |
|---|---|---|
| Phosphatidylcholine (PC) | 69 | 80 |
| Phosphatidylethanolamine | 15 | 8 |
| (PE) | | |
| Sphingomyelin (SPH) | 1 | 2 |
| Lysophosphatidylcholine | 1 | 2 |
| (LPC) | | |
| Phosphorus | 3.6 | 3.8 |

Emulsions were prepared according to 4 different processes:

### Process A (Comparative Process)

The oil phase was provided by heating soybean oil to 78 to 83° C. and then adding the emulsifier under mixing by means of an Ultra-Turrax (T50) for 5 minutes at 5000 rpm.

The water phase was provided by mixing glycerol and water for injection and heating to 78 to 83° C. The pH was adjusted to 9.0 to 10.0 by adding a solution of sodium hydroxide.

The oil phase and the water phase were mixed at 78 to 83° C. by means of an Ultra-Turrax (T50) for 10 to 12 minutes at 10000 rpm. The pH was adjusted to 9.0 to 10.0 by adding a solution of sodium hydroxide.

The pre-emulsion was then homogenized in a high-pressure valve homogenizer in 6 cycles at a pressure of 560 bar in the first stage and at a pressure of 110 bar in the second stage (APV-1000, SPX Flow Technology).

The temperature was maintained between 50 and 60° C. during the homogenization.

After the homogenization the emulsion was cooled to below 30° C. and the pH was adjusted to 9.0 to 10.0.

Finally, the emulsion was sterilized by autoclaving at 121.1° C. for 15 minutes.

### Process B (Comparative Process)

Process B differed from process A only in that the high-pressure homogenization step was performed by means of a counter jet disperser (M-110S; Microfluidics) in 3 cycles at a pressure of 1300 bar.

### Process C (Comparative Process)

Glycerol and 532 ml of water for injection (=water phase 1) were mixed and heated to 78 to 83° C. The pH was adjusted to 9.0 to 10.0 by adding a solution of sodium hydroxide.

The oil phase was provided by heating soybean oil to 78 to 83° C. and then adding the emulsifier under stirring by means of an Ultra-Turrax (T50) for 5 minutes at 5000 rpm.

The oil phase and the water phase 1 were mixed at 78 to 83° C. by means of an Ultra-Turrax (T50) for 10 to 12 minutes at 10000 rpm. The pH was adjusted to 9.0 to 10.0 by adding a solution of sodium hydroxide.

The pre-emulsion was then homogenized in a high-pressure valve homogenizer in 6 cycles at a pressure of 560 bar in the first stage and at a pressure of 110 bar in the second stage (APV-1000, SPX Flow Technology).

The residual water for injection (=water phase 2) was added to adjust the volume of the emulsion to 2 liters and the pH was adjusted to 9.0 to 10.0. Finally, the emulsion was sterilized by autoclaving at 121.1° C. for 15 minutes.

### Process D

Process D differed from process C only in that the high-pressure homogenization step was performed by means of a counter jet disperser (M-110S; Microfluidics) in 3 cycles at a pressure of 1300 bar.

Of the 8 different emulsions theoretical obtainable according to the permutations listed in table 3 the six emulsions marked in bold were prepared and studied as described in example 3:

TABLE 3

| | egg yolk lecithin type + |
|---|---|
| emulsion no. | manufacturing process |
| 1.1 | PL1 + process A |
| 1.2 | PL1 + process B |
| 1.3 | PL1 + process C |
| 1.4 | PL1 + process D |
| 1.5 | PL2 + process A |
| 1.6 | PL2 + process B |
| 1.7 | PL2 + process C |
| 1.8 | PL2 + process D |

### Example 2

Different emulsions were prepared from the ingredients listed in table 4. Their composition only differed in the choice of the emulsifier (egg yolk lecithin) which was either PL1 or PL2.

TABLE 4

| ingredient | amount (g) |
|---|---|
| glycerol | 50.00 |
| emulsifier (either PL1 or PL2) | 24.00 |
| sodium oleate | 0.60 |
| NaOH 1M | q.s. |
| Soybean oil | 120.00 |
| MCT oil | 120.00 |
| olive oil | 100.00 |
| fish oil | 60.00 |
| alpha tocopherol | 0.04 |
| WFI | ad 2000 |
| nitrogen | q.s. |

The emulsions were prepared according to 4 different processes:

### Process A (Comparative Process)

The oil phase was provided by mixing soybean oil, medium chain triglycerides, olive oil, fish oil and alpha tocopherol and heating to 60 to 70° C.

The water phase was provided by mixing glycerol, water for injection and alpha tocopherol, heating to 60 to 70° C. and then adding the emulsifier under continuous stirring.

The oil phase and the water phase were mixed at 62 to 68° C. by means of an Ultra-Turrax (T50) for 10 to 12 minutes at 10000 rpm. The pH was adjusted to 9.0 to 10.0 by adding a solution of sodium hydroxide.

The pre-emulsion was then homogenized in a high-pressure valve homogenizer in 6 cycles at a pressure of 560 bar in the first stage and at a pressure of 120 bar in the second stage (APV-1000, SPX Flow Technology). The temperature was maintained between 50 and 60° C. during the homogenization.

After the homogenization the emulsion was cooled to below 30° C. and the pH was adjusted to 9.0 to 10.0.

Finally, the emulsion was sterilized by autoclaving at 121° C. for 15 minutes.

### Process B (Comparative Process)

Process B differed from process A only in that the high-pressure homogenization step was performed by means of a counter jet disperser (M-110S; Microfluidics) in 3 cycles at a pressure of 900 bar.

### Process C (Comparative Process)

Glycerol, sodium oleate and 325 ml of water for injection were mixed and heated to 60 to 70° C. Then, the emulsifier was added under stirring by means of an Ultra-Turrax (T50) at 5000 rpm for 5 minutes to obtain the water phase 1.

The pH of the water phase 1 was adjusted to 9.0 to 10.0 by adding a solution of sodium hydroxide.

The oil phase was provided by mixing the four oils and alpha tocopherol. The oil phase was heated to 60 to 70° C.

The oil phase and the water phase 1 were mixed at 60 to 70° C. by means of an Ultra-Turrax (T50) for 10 to 12 minutes at 10000 rpm. The pH was adjusted to 9.0 to 10.0 by adding a solution of sodium hydroxide.

The pre-emulsion was then homogenized in a high-pressure valve homogenizer in 6 cycles at a pressure of 560 bar in the first stage and at a pressure of 120 bar in the second stage (APV-1000, SPX Flow Technology).

The residual water for injection (=water phase 2) was added to adjust the volume of the emulsion to 2 liters, and the pH was adjusted to 9.0 to 10.0.

Finally, the emulsion was sterilized by autoclaving at 121.1° C. for 15 minutes.

### Process D

Process D differed from process C only in that the high-pressure homogenization step was performed by means of a counter jet disperser (M-110S; Microfluidics) in 3 cycles at a pressure of 900 bar.

Of the 8 different emulsions theoretical obtainable according to the permutations listed in table 5 the five emulsions marked in bold were prepared and studied as described in example 3.

TABLE 5

| emulsion no. | composition and manufacturing process |
|---|---|
| 2.1 | PL1 + process A |
| 2.2 | PL1 + process B |
| 2.3 | PL1 + process C |
| 2.4 | PL1 + process D |
| 2.5 | PL2 + process A |
| 2.6 | PL2 + process B |
| 2.7 | PL2 + process C |
| 2.8 | PL2 + process D |

### Example 3

The emulsions prepared according to examples 1 and 2 were mixed with different amino acid solutions for parenteral administration (column 2) and with glucose solutions for parenteral administration of different glucose concentrations (column 3) as listed in table 6.

First, the amino acid solution and the glucose solution were mixed in the ratio according to table 6 (column 4). Then the oil-in-water emulsion obtained according to example 1 or 2 respectively was added in the appropriate amount to obtain the ratio according to table 6.

The pH values (column 5) and the osmolarities (column 6) of the resulting mixtures are also listed in table 6.

The PFAT₅ values of the resulting mixtures were measured directly after their preparation (t0), after 24 hours (t1) and after 48 hours (t2) respectively according to USP <729> (method 2) by means of an AccuSizer 780.

TABLE 6

| | | | ratio emulsion to | | |
|---|---|---|---|---|---|
| | amino acid | glucose | glucose solution | | |
| Experiment | solution (AA | concentration | to AA solution | | osmolarity |
| no. | concentration) | [%] | (v:v:v) | pH | [mOsmol/L] |
| 1 | 1 (10%) | 13 | 1:3.86:2.24 | 5.6 | 950 |
| 2 | 1 (10%) | 42 | 1:2.67:1.59 | 5.6 | 1500 |
| 3 | 2 (11.4%) | 19 | 1:2.63:1.50 | 5.6 | 1060 |
| 4 | 2 (11.4%) | 11 | 1:3.47:1.18 | 5.6 | 750 |
| 5 | 1 (10%) | 42 | 1:1.59:2.66 | 5.6 | 1500 |
| 6 | 1 (10%) | 13 | 1:3.86:2.23 | 5.6 | 850 |
| 7 | 1 (10%) | 42 | 1:1.40:4.53 | 5.6 | 1300 |
| 8 | 1 (10%) | 13 | 1:3.28:1.43 | 5.6 | 750 |
| 9 | 3 (6.53%) | 22 | 1:6.93:6.06 | 5.2 | 840 |
| 10 | 3 (6.53%) | 20 | 1:4.76:3.57 | 5.8 | 900 |
| 11 | 3 (6.53%) | 18 | 1:5.29:2.95 | 5.5 | 880 |

Experiment 1 was performed by mixing 47 ml of the amino acid solution 1, 82 ml of the glucose solution, and 21 ml of the emulsion (obtaining 150 ml of the mixture).

Experiment 4 was performed by mixing 31 ml of the amino acid solution 2, 92 ml of glucose solution, and 27 ml of the emulsion to (obtaining 150 ml of the mixture). After mixing the amino acid solution, the glucose solution and the emulsion, the resulting mixtures were stored in Freeflex® bags at room temperature for 48 hours.

Amino acid solution 1 has a pH of 5.5 to 6.3, a theoretical osmolarity of 990 mOsmol/L and comprises in 1000 ml:
L-Isoleucine 5.00 g; L-Leucine 7.40 g; L-Lysine acetate 9.31 g (=6.60 g L-Lysine); L-Methionine 4.30 g; L-Phenylalanine 5.10 g; L-Threonine 4.40 g; L-Tryptophan 2.00 g; L-Valine 6.20 g; L-Arginine 12.00 g; L-Histidine 3.00 g; L-Alanine 14.00 g; Glycine 11.00 g; L-Proline 11.20 g; L-Serine 6.50 g; L-Tyrosine 0.40 g; Taurine 1.00 g

Amino acid solution 2 has a pH of 5.6, an osmolarity of 1130 mOsmol/L and comprises in 1000 ml:
Glycine 7.9 g; L-Aspartic acid 3.4 g; L-Glutamic acid 5.6 g; L-Alanine 16.0 g; L-Arginine 11.3 g; L-Cysteine 560 mg; L-Histidine 6.8 g; L-Isoleucine 5.6 g; L-Leucine 7.9 g; L-Lysine acetate (expressed as L-Lysine) 9.0 g; L-Methionine 5.6 g; L-Phenylalanine 7.9 g; L-Proline 6.8 g; L-Serine 4.5 g; L-Threonine 5.6 g; L-Tryptophan 1.9 g; L-Tyrosine 230 mg; L-Valine 7.3 g

Amino acid solution 3 has a pH of 5.2, an osmolarity of 510 mOsmol/L and comprises in 1000 ml:
L-Alanine 6.3 g; L-Arginine 4.1 g; L-Aspartic acid 4.1 g; L-Cysteine (+L-Cystine) 1.0 g; L-Glutamic acid 7.1 g; Glycine 2.1 g; L-Histidine 2.1 g; L-Isoleucine 3.1 g; L-Leucine 7.0 g; L-Lysine monohydrate (corresponding to L-Lysine) 5.6 g; L-Methionine 1.3 g; L-Phenylalanine; 0.7 g; L-Proline 5.6 g; L-Serine 3.8 g; Taurine 300 mg; L-Threonine 3.6 g; L-Tryptophan 1.4 g; L-Tyrosine 500 mg; L-Valine 3.6 g

### Results

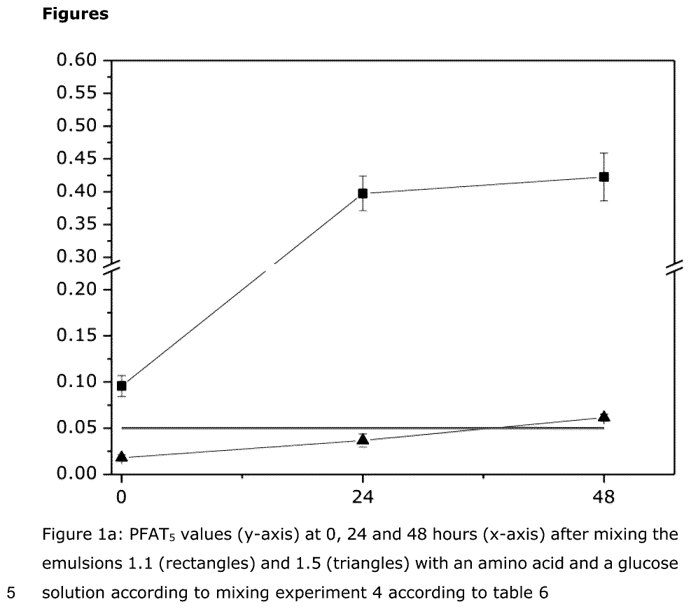

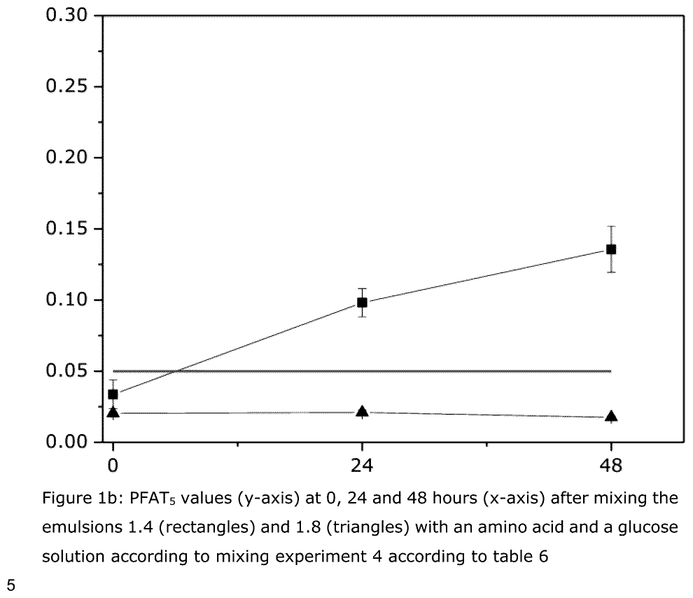

In order to study the influence of the emulsifier, emulsions 1.1 (prepared via preparation method A comprising PL1) and 1.5 (prepared via preparation method A comprising PL2) were studied according to admixing experiment 4 as listed in table 6 and the PFAT₅ values were compared. The PFAT₅ values are depicted in FIG. 1a. Emulsion 1.5 had the lower PFAT₅ value at t0, t1 and t2. However, also the PFAT₅ value of emulsion 1.5 exceeded 0.05% after 48 hours (at t2).

Further, emulsion 1.4 (prepared via preparation method D comprising PL1) and emulsion 1.8 (prepared via preparation method D comprising PL2) were studied according to admixing experiment 4 as listed in table 6 and the PFAT₅ values were compared. The PFAT₅ values are depicted in FIG. 1b. Emulsion 1.8 had the lower PFAT₅ value at t0, t1 and t2. The PFAT₅ value of emulsion 1.8 did not exceed 0.05% at t0, t1 and t2. By contrast, the PFAT₅ value of emulsion 1.4 exceeded 0.05% already at t0.

Hence, the choice of the emulsifier is crucial. However, choosing an emulsifier with a phosphatidyl choline content of at least 70% based on the total weight of the emulsifier alone is not sufficient to obtain emulsions with PFAT₅ values that remain below 0.05% for at least 48 hours upon admixture of the emulsion with an amino acid suitable for parenteral administration and a glucose solution suitable for parenteral administration.

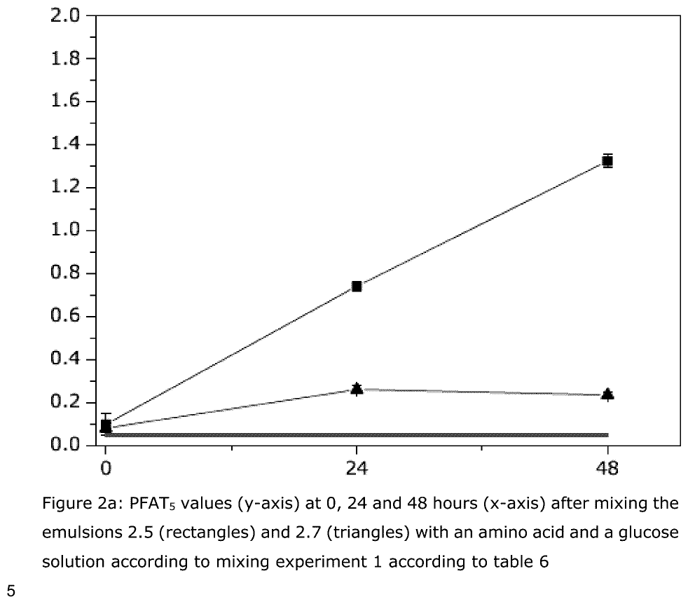

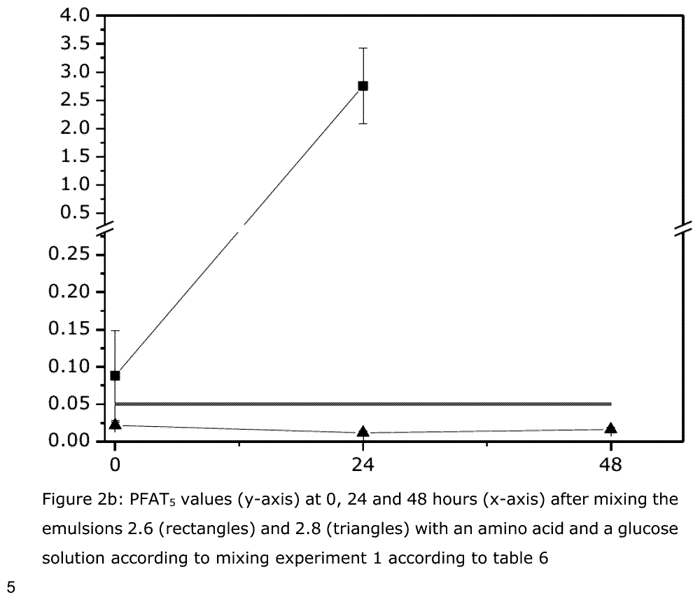

In order to study the influence of the concentration of the oil phase during homogenization, emulsion 2.5 (prepared via preparation method A comprising PL2) and emulsion 2.7 (prepared via preparation method C comprising PL2) were studied according to admixing experiment 1 as listed in table 6 and the PFAT₅ values were compared. The PFAT₅ values are depicted in FIG. 2a. Emulsion 2.7 had the lower PFAT₅ value at t0, t1 and t2. However, the PFAT₅ value of both emulsions exceeded 0.05% already at to.

Further, emulsion 2.6 (prepared via preparation method B comprising PL2) and emulsion 2.8 (prepared by preparation method D comprising PL2) were studied according to admixing experiment 1 as listed in table 6 and the PFAT₅ values were compared. The PFAT₅ values are depicted in FIG. 2b. The PFAT₅ value of emulsion 2.8 was below 0.05% at t0, t1 and t2. By contrast, the PFAT₅ value of emulsion 2.6 exceeded 0.05% already at to.

Hence, the concentration of the oil phase during homogenization is crucial. However, a higher concentration of oil phase alone is not sufficient to obtain emulsions with PFAT₅ values that remain below 0.05% for at least 48 hours upon admixture of the emulsion with an amino acid suitable for parenteral administration and a glucose solution suitable for parenteral administration.

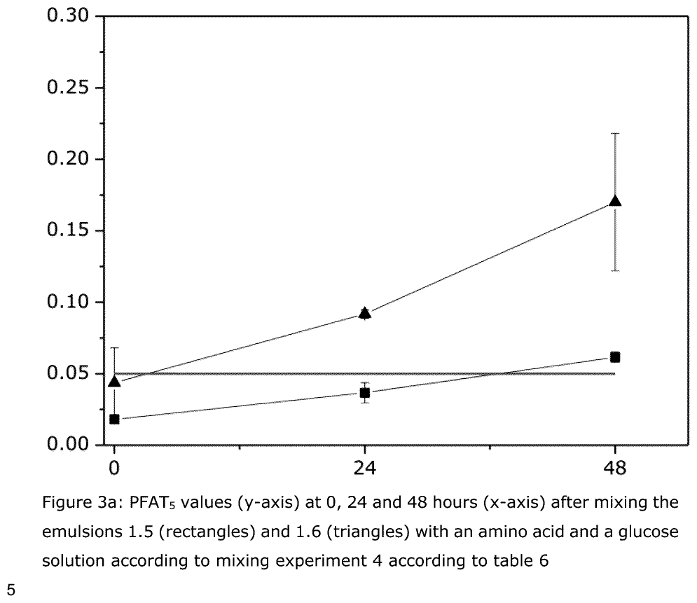

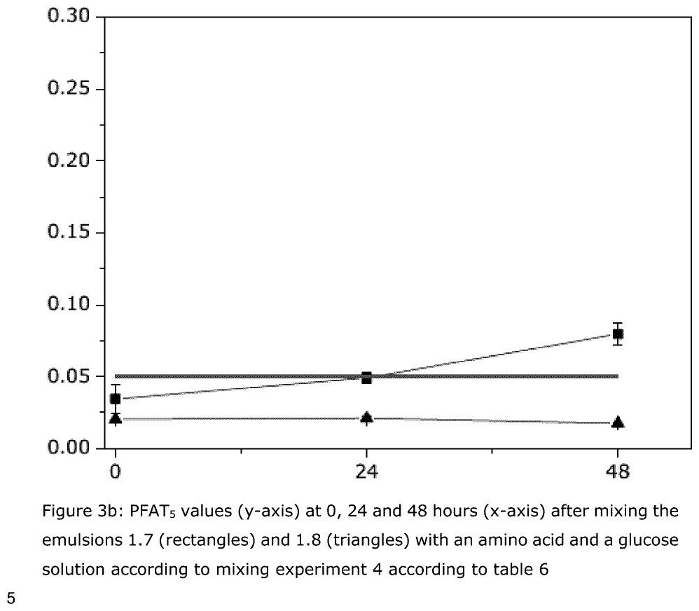

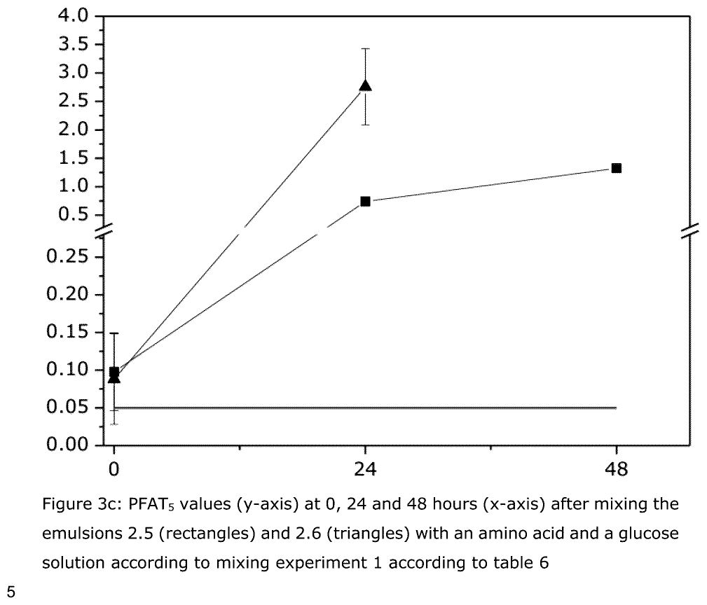

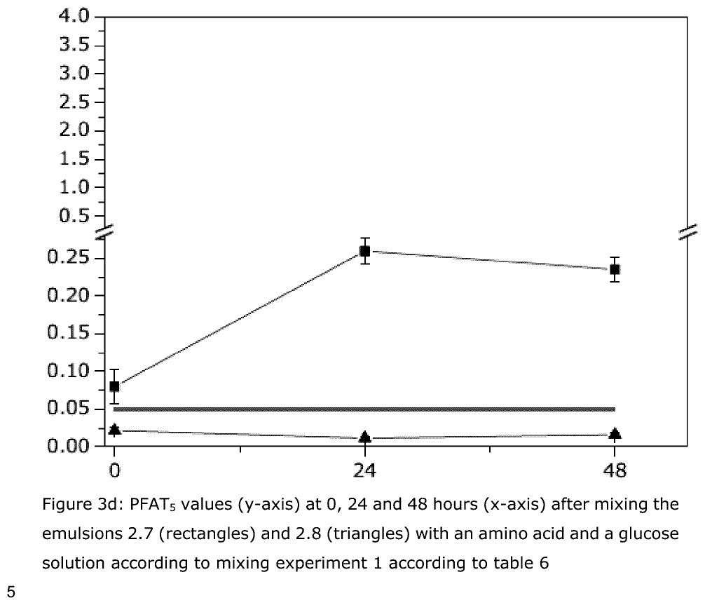

In order to study the influence of the homogenization technique, emulsions 1.5 (prepared according to preparation method A comprising PL2) and 1.6 (prepared according to preparation method B comprising PL2) were studied according to admixing experiment 4 as listed in table 6 and the PFAT₅ values were compared. The results are depicted in FIG. 3a. The PFAT₅ value of emulsion 1.6 was lower than the PFAT₅ value of emulsion 1.5 at t0, t1 and t2. However, also the PFAT₅ value of emulsion 1.6 exceeded 0.05% at t2.

Also, emulsion 1.7 (prepared according to preparation method C comprising PL2) and emulsion 1.8 (prepared according to preparation method D comprising PL2) were studied according to admixing experiment 4 as listed in table 6 and the PFAT₅ values were compared.

The results are depicted in FIG. 3b. The PFAT₅ value of emulsion 1.8 was below 0.05% and lower than the PFAT₅ value of emulsion 1.7 at t0, t1 and t2. The PFAT₅ value of emulsion 1.7 exceeded 0.05% at t2.

To further study the influence of the homogenization technique, emulsions 2.5 (prepared according to preparation method A comprising PL2) and 2.6 (prepared according to preparation method B comprising PL2) were studied according to admixing experiment 1 as listed in table 6 and the PFAT₅ values were compared. The results are depicted in FIG. 3c. The PFAT₅ value of emulsion 2.6 was lower than the PFAT₅ value of emulsion 2.5 at t1. However, the PFAT₅ value of both emulsions exceeded 0.05% already at to.

Also, emulsion 2.7 (prepared according to preparation method C comprising PL2) and emulsion 2.8 (prepared according to preparation method D comprising PL2) were studied according to admixing experiment 1 as listed in table 6 and the PFAT₅ values were compared. The results are depicted in FIG. 3d. The PFAT₅ value of emulsion 2.8 was below 0.05% and lower than the PFAT₅ value of emulsion 2.7 at t0, t1 and t2. The PFAT₅ value of emulsion 2.7 exceeded 0.05% already at to.

Hence, the homogenization technique is crucial. However, performing the homogenization by means of a counter jet disperser alone is not sufficient to obtain emulsions with PFAT₅ values that remain below 0.05% for at least 48 hours upon admixture of the emulsion with an amino acid suitable for parenteral administration and a glucose solution suitable for parenteral administration.

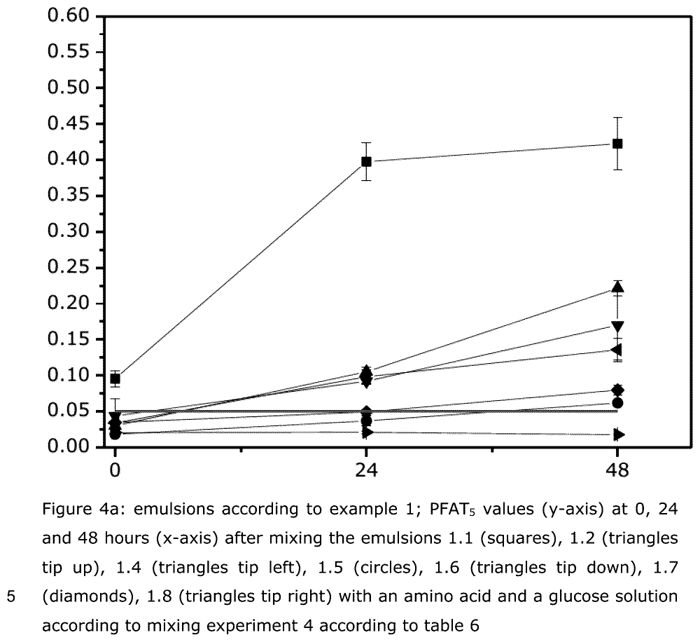

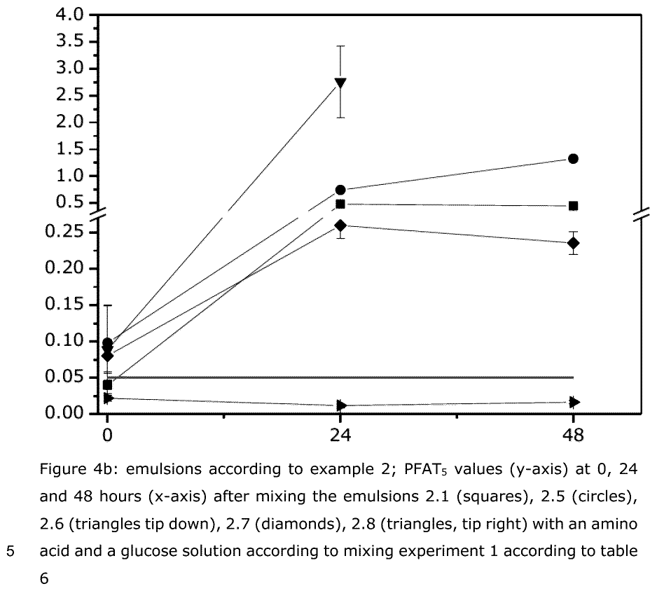

These findings are summarized in FIGS. 4a and 4b. In FIG. 4a the PFAT₅ values of the emulsions according to example 1 are compared. In FIG. 4b the emulsions according to example 2 are compared. In both cases, only the PFAT₅ value of the emulsion prepared according to preparation method D and comprising PL2 (emulsions 1.8 and 2.8 respectively) remained below 0.05% for at least 48 hours upon admixture with an amino acid suitable for parenteral administration and a glucose solution suitable for parenteral administration. Emulsions 1.8 and 2.8 were subjected to all admixing experiments listed in table 6.

The PFAT₅ values were below 0.05% at t0, t1 and t2. Hence, the process of the present invention ensures that the PFAT₅ values of the emulsions obtained according to the process remain below 0.05% for at least 48 hours upon admixture with different amino acid solutions suitable for parenteral administration, different glucose solutions suitable for parenteral administration, at different pH values and at different osmolarities respectively.

1. A process for manufacturing an oil-in-water emulsion, said process comprising a water phase and 5 to 25 wt. % of an oil phase based on the total weight of the emulsion, the process comprising the following steps:
(a) providing an oil phase comprising one or more oils selected from the group consisting of animal derived oils, plant-derived oils, fungal oils, synthetic or semi-synthetic fatty acid triglycerides, microbial oils and algae oils,
(b) providing an aqueous phase 1 comprising water,
(c) obtaining a pre-emulsion by mixing the oil phase provided in step a) with the aqueous phase 1 provided in step b),
(d) obtaining a first emulsion by homogenizing the pre-emulsion obtained in step c) by means of at least one counter-jet disperser, wherein the homogenization is performed in 2-6 cycles, at a pressure of 600-1800 bar and at a temperature of 40-80° C.,
(e) providing an aqueous phase 2 comprising water,
(f) obtaining the oil-in-water emulsion by mixing the first emulsion obtained in step d) with the aqueous phase 2 provided in step e) and
(g) sterilizing the oil-in-water emulsion obtained in step f) and filling it into a suitable container either before or after sterilization,
wherein in step a) or b) a pharmaceutically acceptable emulsifier, characterized in that it comprises at least 70 wt. % phosphatidyl choline based on the total weight of the emulsifier, is added,
wherein in the pre-emulsion obtained in step c) and in the first emulsion obtained in step d) the concentration of the oil phase is 130 to 350% of the concentration of the oil phase in the oil-in-water emulsion obtained in step f),
and wherein for at least 24 hours, preferably 48 hours, after mixing the emulsion with an amino acid solution suitable for parenteral administration and/or a glucose solution suitable for parenteral administration the PFAT₅ value of the resulting mixture does not exceed 0.05%.

2. The process according to claim 1, wherein in the pre-emulsion obtained in step c) and in the first emulsion obtained in step d) the concentration of the oil phase is 150 to 350%, preferably 180 to 330%, more preferably 200 to 300% of the concentration of the oil phase in the oil-in-water emulsion obtained in step f).

3. The process according to claim 1, wherein the oil phase comprises one or more oils selected from the group consisting of medium chain triglycerides, structured lipids, almond oil, coconut oil, cotton seed oil, linseed oil, olive oil, palm kernel oil, peanut oil, rapeseed oil, safflower oil, sesame oil, soybean oil, sunflower oil, fish oil, fish oil extract and krill oil, preferably medium chain triglycerides, structured lipids, olive oil, soybean oil, fish oil or mixtures thereof.

4. The process according to claim 1, wherein the oil phase comprises soybean oil and wherein in the first emulsion obtained in step d) the concentration of the oil phase is 180 to 350%, preferably 180 to 330%, more preferably 180 to 300%, of the concentration of the oil phase in the oil-in-water emulsion obtained in step f).

5. The process according to claim 1, wherein the oil phase comprises soybean oil, MCT, olive oil and fish oil and wherein in the first emulsion obtained in step d) the concentration of the oil phase is 180 to 350%, preferably 180 to 330%, more preferably 200 to 300%, of the amount of the oil phase in the oil-in-water emulsion obtained in step f).

6. The process according to claim 1, wherein in step b) and/or in step e) at least one pharmaceutically acceptable co-surfactant and/or a pH adjusting agent and/or a pharmaceutically acceptable preservative and/or at least one pharmaceutically acceptable tonicity agent is added.

7. The process according to claim 1, wherein in step a) a pharmaceutically acceptable antioxidant and/or a pharmaceutically acceptable co-surfactant is added.

8. The process according to claim 1, wherein for at least 24 hours, preferably at least 48 hours, after mixing the emulsion with an amino acid solution suitable for parenteral administration and a glucose solution suitable for parenteral administration the PFAT₅ value of the resulting mixture does not exceed 0.05%, preferably 0.04%, more preferably 0.03%.

9. The process according to claim 1, wherein the pharmaceutically acceptable emulsifier is characterized in that it comprises at least 72 wt. %, preferably at least 74 wt. %, more preferably at least 76 wt. %, most preferably at least 78 wt. %, phosphatidyl choline based on the total weight of the emulsifier.

10. The process according to claim 1, wherein the pharmaceutically acceptable antioxidant is tocopherol, preferably alpha tocopherol or a mixture of alpha tocopherol, beta tocopherol and gamma tocopherol.

11. The process according to claim 6, wherein the pharmaceutically acceptable co-surfactant is oleic acid or sodium oleate.

12. The process according to claim 6, wherein the pharmaceutically acceptable tonicity agent is glycerol.

13. The process according to claim 1, wherein the homogenization in step d) is performed in 3 to 4 cycles at a pressure of 800 to 1400 bar and a temperature of 45 to 65° C.

14. The process according to claim 1, wherein the oil phase comprises soybean oil and wherein homogenization in step d) is performed in 3-4 cycles at a pressure of 1200 to 1400 bar and a temperature of 45 to 65° C., preferably 50 to 60° C.

15. The process according to claim 1, wherein the oil phase comprises soybean oil, medium chain triglycerides, olive oil and fish oil and wherein the homogenization in step d) is performed in 3 to 4 cycles at a pressure of 800 to 1000 bar and a temperature of 45 to 65° C., preferably 50 to 60° C.

16. An oil-in-water Oil in water emulsion for providing parenteral nutrition obtained by the process according to claim 1.

17. (canceled)
